(19) United States
(12) Patent Application Publication
(10) Pub. No.: US 2008/0081026 A1
(43) Pub. Date: Apr. 3, 2008

(54) Cosmetic product

(75) Inventors: Kojo Tanaka, Ashiya (JP); Minako Yoshimi, Nishinomiya (JP); Keiko Tanii, Amagasaki (JP); Yasuko Suzuki, Kobe (JP); Iku Matsubara, Kobe (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/904,527

(22) Filed: Sep. 27, 2007

Related U.S. Application Data
(60) Provisional application No. 60/847,804, filed on Sep. 28, 2006.
(60) Provisional application No. 60/861,936, filed on Nov. 30, 2006.

(51) Int. Cl. A61K 8/27; A61K 8/29; A61Q 1/02

(52) U.S. Cl. 424/63

(57) ABSTRACT

Disclosed is a cosmetic product comprising: (a) a foundation comprising less than about 15% by weight of the foundation of a high coverage pigment selected from the group consisting of titanium dioxide, zinc oxide, and mixtures thereof; and (b) a concealer comprising at least about 25% by weight of the concealer of a high coverage pigment selected from the group consisting of titanium dioxide, zinc oxide, and mixtures thereof; wherein; (c) the CMC DE of the foundation color and the concealer color are less than 1.5; and (d) the foundation and concealer are of the same product form.

### CROSS REFERENCE TO RELATED APPLICATIONS

This application claims the benefit of U.S. Provisional Application No. 60/847,804 filed on Sep. 28, 2006; and U.S. Provisional Application No. 60/861,936 filed on Nov. 30, 2006.

### FIELD OF THE INVENTION

The present invention relates to a cosmetic product comprising a foundation and concealer, wherein the foundation and concealer are of the same color, and are further of the same product form. The present invention further relates to methods of make-up using such cosmetic product. The present invention further relates to cosmetic products which contain such foundation and concealer in the same package. The present invention further relates to a personal care product comprising multiple applicators.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Concealers are used to obtain high coverage on a particularly concerned area, and supplement the function of a foundation on. Concealers are typically in the form of liquid, paste, or semi-solid and contain a high level of pigments having opacity, such as titanium dioxide, and are typically used prior to applying the foundation.

One of the unsatisfying points for concealer users is that, the portion of skin for which the concealer is applied provides an unnatural appearance. Namely, in view of different texture or different color provided by the concealer, the skin area on which concealer is applied is noticeable. Considering that concealers are used in desire to make a certain concerned area of the skin to be less conspicuous from the surrounding area, such unnatural appearance is contrary to what is desired.

Another unsatisfying point for concealer users is that, concealer products are typically in liquid form and are designed to apply to the skin prior to application of foundation. Thus, concealer products are inconvenient to carry, and inconvenient to use as touch-up during the day.

Cosmetic kit products providing compositions of different formulation are disclosed in, for example, Japanese Patent Publication H10-87471, 2002-114638, 2000-281529, 10-87471, 6-157263, 6-145022, 6-128122, and 5-58840. There are no references, however, which recognize the benefit of providing the concealer with the same shade as the foundation with which it is to be used.

Based on the foregoing, there is a need for a concealer which, in combination with a foundation, can naturally cover skin imperfections and provide a seamless appearance. There is further a need to provide a method of make-up which provides improved natural coverage to skin imperfections by way of usage of a concealer. There is further a need to provide a concealer in a product form that is convenient to use and carry. None of the existing art provides all of the advantages and benefits of the present invention.

### SUMMARY

The present invention is directed to a cosmetic product comprising:

(a) a foundation comprising less than about 15% by weight of the foundation of a high coverage pigment selected from the group consisting of titanium dioxide, zinc oxide, and mixtures thereof; and

(b) a concealer comprising at least about 25% by weight of the concealer of a high coverage pigment selected from the group consisting of titanium dioxide, zinc oxide, and mixtures thereof; wherein;

(c) the CMC DE of the foundation color and the concealer color are less than 1.5; and

(d) the foundation and concealer are of the same product form.

The present invention is also directed to a method of making up the skin utilizing the aforementioned cosmetic product.

The present invention is further directed to a personal care product comprising a composition for care of a personal surface, multiple applicators for applying the composition to a personal surface, and other elements of convenience to the user.

These and other features, aspects, and advantages of the present invention will become evident to those skilled in the art from a reading of the present disclosure with the appended claims.

### BRIEF DESCRIPTION OF THE DRAWINGS

While the specification concludes with claims particularly pointing out and distinctly claiming the invention, it is believed that the present invention will be better understood from the following description of preferred, nonlimiting embodiments and representations taken in conjunction with the accompanying drawings in which:

FIG. 1 is a schematic view of a preferred embodiment of a package suitable for use with the product of the present invention.

FIG. 2 is a schematic view of a preferred embodiment of a replaceable applicator tray suitable for use with the product of the present invention.

### DETAILED DESCRIPTION OF THE INVENTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Foundation and Concealer

The present cosmetic product comprises a foundation comprising less than about 15% by weight of the foundation of high coverage pigment, and a concealer comprising at least about 25% by weight of the concealer of high coverage pigment. Herein, a foundation is a composition which provides natural coverage of the skin, and has a certain color resembling the color of the skin. The function of a foundation therefore is to even the skin tone and texture of the skin to which it is applied, typically to the entire surface of the facial skin. Herein, a concealer is a composition which provides higher coverage than a foundation, and is typically used to a particular area of the skin for which specific care of appearance is desired. Characteristic of the concealer of the present invention is that the concealer also has a color. The difference in amount of high coverage pigment in the foundation and concealer compositions provides such difference in coverage. The color of the foundation typically resembles the facial skin. The concealer also has color, wherein the CMC DE of the foundation color and the concealer color are less than 1.5. Accordingly, there is substantially no difference in color of the foundation and concealer in the eyes of the general consumer. The color of the foundation and concealer compositions can be created by color powders as described hereinbelow, dyes, and other colorant materials.

The present foundation and concealer can be of any product form, including loose powder, pressed powder, semi-solid, paste, and liquid. The product form of the foundation and the concealer are the same. Without being bound by theory, it is believed that, by matching the product form of the foundation and concealer, the texture of the two compositions, when applied to the skin, are very similar, such that the concealer provides natural coverage to the portion of the skin where higher coverage is needed. Preferably, the foundation and concealer are both liquid form or both powder form. Powder product forms typically have a total powder content of at least about 75%. Liquid product forms typically have a total powder content of no more than about 50%. It is possible to provide the foundation and concealer in product forms having a total powder content of between about 50% and about 75%. It is preferred, however, to provide both compositions in either both a liquid form, or both a powder form.

The foundation and concealer of the present invention have substantially no difference in color, and are of the same product form. By having such common color and product form, the present product provides a seamless appearance to the skin, namely, the portion where concealer were applied is not recognizable.

In one preferred embodiment, the foundation and concealer are both powder form, and are provided in the same package.

The present foundation and concealer compositions further contain an appropriate carrier for providing the desired product form. Such carrier may include non-volatile oil, lipophilic surfactant, humectant, water, volatile silicone oil, and thickeners such as fatty compound, solid wax, gelling agent, inorganic thickening agent, and silicone elastomer. For providing a powder form, the powders are mixed with a binder comprising one or more of non-volatile oil, lipophilic surfactant, and humectant. For providing an emulsified composition, non-volatile oil, liphophilic surfactant, water, and optional surfactant and thickeners may be used. Thickeners may be used to control viscosity or solidify the composition. Volatile silicone oils may be used for providing freshness to the skin, and favorably used in liquid product forms.

### High Coverage Pigment

The present cosmetic product comprises a foundation comprising less than about 15% by weight of the foundation of a high coverage pigment, and a concealer comprising at least about 25% by weight of the concealer of a high coverage pigment. Preferably, the amount of high coverage pigment is at least about 25% and no more than about 50% of the concealer, for providing a natural look.

The high coverage pigment herein is selected from the group consisting of titanium dioxide, zinc oxide, and mixtures thereof, and have an average particle size of from about 100 nm to about 500 nm, preferably from about 200 nm to about 350 nm. The titanium dioxide may be rutile type or anatase type. For the concealer, the high coverage pigment is preferably at least partially used as complex pigments with other organic/inorganic components, and are also preferably hydrophobically coated. For calculating the amount of high coverage pigment of the complex pigments, only the high coverage pigment content or coating is counted.

Useful complexed pigments for the concealer include titanium coated pigment made of a core pigment selected from the group consisting of talc, mica, sericite, synthetic mica, aluminum oxide, silica, boron nitride, and mixtures thereof, which core pigment is coated with titanium dioxide having a particle size of from about 100 nm to about 500 nm, and the titanium dioxide coating accounting for at least 33% of the titanium coated pigment, preferably at least 40% of the titanium coated pigment. By such higher coating, such titanium coated pigments provide a high coverage effect compared to the same weight of pigments made solely of titanium dioxide.

Commercially available high coverage pigments highly useful herein include 57.3% titanium dioxide coated sericite further coated with methicone in the tradename of Fancyveil S-3060SW available from Shokubai Kasei, and Titanium Dioxide coated with Methicone with tradename SI Titanium Dioxide IS available from Miyoshi Kasei.

### Other Powders

The present foundation and concealer compositions contain powders other than the high coverage pigment to provide a color resembling the skin tone. The other powders herein may also provide other appearance and skin feel effects. For providing products of powder form, the total powder content is at least about 75% of the foundation composition or the concealer composition. For providing products of liquid form, the total powder content is no more than about 50% of the foundation composition or the concealer composition. Herein, total powder means any powder component including the high coverage pigment.

In the present invention, the powders are selected such that the color of the foundation and concealer are substantially the same. It is within ordinary skill of the art to provide similar color to compositions having different content of high coverage pigment.

The other powders useful herein include those that provide color or change tone, and also those that provide a certain skin feel. Useful powders herein include clay mineral powders such as talc, mica, sericite, bentonite and montomorilonite; coloring powders useful herein include iron oxides, iron titate, ultramarine blue, Prussian blue, chromium oxide, chromium hydroxide, cobalt oxide, cobalt titanate, titanium oxide coated mica, laked tar color dyes, and laked natural color dyes; organic powders herein include polyacrylates such as methyl methacrylate copolymer and methyl methacrylate crosspolymers, celluloses, polyalkylenes such as polyethylene and polypropylene, vinyl acetates, polystyrenes such as styrene-acrylic acid copolymers, polyamides such as nylon-12 and nylon-6, acrylic acid ethers such as acrylic acid methyl ether and acrylic acid ethyl ether, polyvinyl pyrrolidones; vinyl chloride polymers, silicones such as polyorganosilsesquioxane resin and solid silicone elastomers, tetrafluoroethylene polymer, and fish scale guanine; inorganic powders such as barium sulfate, calcium secondary phosphate, hydroxy apatite, silicates; such as calcium silicate, magnesium silicate, barium silicate and aluminium silicate, silica beads, metal dioxides such as zirconium oxide and aluminium hydroxide, carbonates such as calcium carbonate and magnesium carbonate, boron nitride and synthetic fluorphlogopite.

In one preferred embodiment, polyorganosilsesquioxane resin and solid silicone elastomers may be used in either the foundation or concealer for enhancing the effect of hiding skin pores.

In another preferred embodiment, the concealer may comprise a metal soap coated pigment made of a core pigment selected from the group consisting of sericite, talc, mica, titanium dioxide, synthetic mica, aluminum oxide, silica, boron nitride, and mixtures thereof, which core pigment is coated with one or more metal soap, such as aluminum dimyristate, aluminium stearate, magnesium stearate, zinc myristate, magnesium myristate, zinc palmitate, zinc laurate, calcium stearate, and mixtures thereof, the metal soap coating accounting for at least 1% of the metal soap coated pigment, preferably at least 3% of the metal soap coated pigment. By such higher coating, such metal soap coated pigment provides good adhesion between itself and the skin, as well as between the pigments themselves.

The powders herein may be surface coated with a coating material having hydrophobic characteristics, or lipophobic hydrophobic characteristics. Useful hydrophobic coating materials herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof. Useful lipophobic hydrophobic coating materials are fluorine compounds such as perfluorooctyl triethoxylsilane, perfluoroalkylphosphoric acids, their salts, and mixtures thereof.

Commercially available powders highly useful herein include methyl methacylate copolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, vinyl dimethicone/methicone silsesquioxane crosspolymer with tradenames KSP series available from ShinEtsu Chemical Co., Ltd., Tokyo Japan, hardened polyorgano siloxane elastomers with tradenames TREFIL series available from Toray Dow Corning, boron nitride with tradename SHP series available from Mizushima Ferroalloy Co., Ltd., 5% aluminum dimyristate coated mica in the name of 5MI MICA M-102, 5% aluminum dimyristate coated sericite in the name of 5MI SERICITE, and 5% aluminum dimyristate coated talc in the name of 5MI TALC JA-46R, all available from Miyoshi Kasei.

### Package

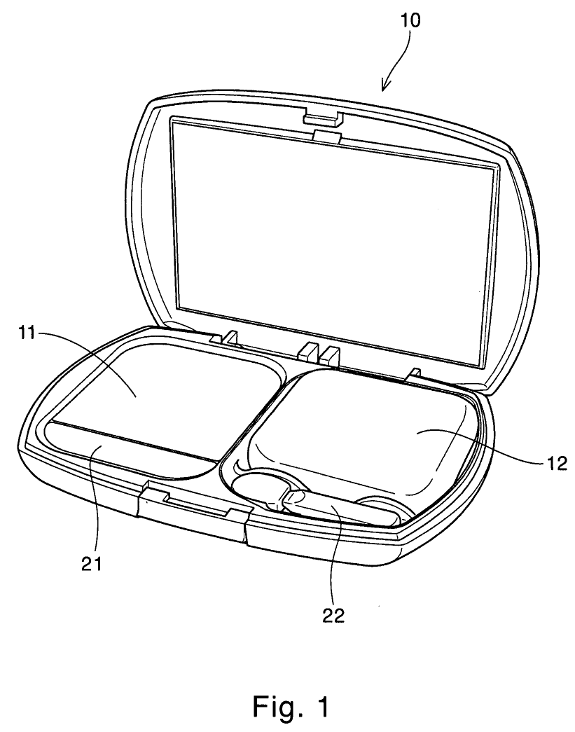

In one preferred embodiment, the foundation and concealer are both powder form, and are provided in the same package. FIG. 1 depicts a preferred embodiment for such package. The package is a compact 10 housing a pan for the foundation 11, a pan for the concealer 21, and an applicator. Preferably, two applicators are contained in the package such that the foundation and concealer can be used via separate applicators. Suitable for foundation is a sponge 12 for broad application, and suitable for concealer is a tip 22 for focused application.

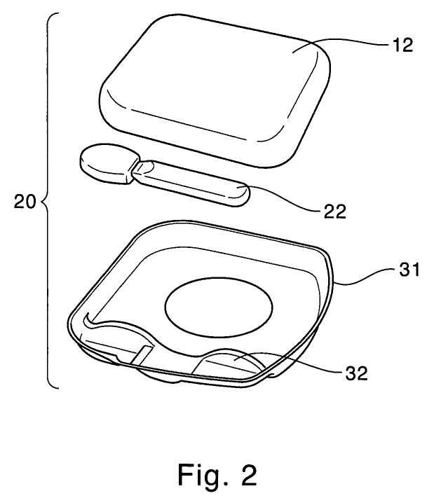

In yet a preferred embodiment, the applicators may be provided in a replaceable tray. It is a habit of prestige segment consumers to re-use the compact upon consumption of a foundation by replacing the used foundation pan with a fresh one. It is also common for such consumers to replace the used applicator with a fresh one at the same time. In the present package wherein more than one applicator is used, it is convenient to provide the applicators in one replaceable tray, such that the consumer may replace the applicators at once, rather than one at a time. FIG. 2 depicts such a replaceable applicator tray 31. The tray 31 has a wall 32 for keeping the sponge 12 and tip 22 in their designated positions, enhancing the convenience to the consumer. The replaceable applicator tray, along with the applicators, may be provided as a kit 20.

In another aspect, the present invention is related to a personal care product comprising:

(a) a composition for care of a personal surface;

(b) multiple applicators for applying the composition to a personal surface; and

(c) a replaceable tray for housing the multiple applicators;

wherein the above mentioned elements are provided in the same package.

In another aspect, the present invention is related to a personal care product comprising:

(a) a composition for care of a personal surface;

(b) multiple applicators for applying the composition to a personal surface; and

(c) a dividing means for keeping each of the multiple applicators in a designated position;

wherein the above mentioned elements are provided in the same package.

The personal care product herein may be any that is suitably applied with an applicator including, but not limited to, facial cosmetic products such as foundation, concealer, blusher, eye shadow, eye brow, eye liner, rouge for lips, and highlighter; skin care products; hair dressing products; and health care products.

The personal care product of the present invention may have the same, or different types of applicators for applying the composition on a personal surface. Multiple applicators may be provided for the purpose of applying the personal care composition to different parts of the personal surface, or for using different types or colors of personal care compositions, or single usage for hygiene purpose.

The multiple applicators may be provided in a replaceable tray, which engages with the package, for housing the multiple applicators. In this way, the multiple applicators may be replaced easily. The applicators may be desired for replacement after one or a certain number of usages, or upon replacement of the personal care composition. One such embodiment for a foundation and concealer product is described above.

The multiple applicators may be provided in a package comprising a dividing means for keeping each of the multiple applicators in a designated position. The dividing means may be a wall embodiment such as described above for a foundation and concealer product. The dividing means prevents the multiple applicators from bunching up or being significantly misplaced from its desired position, and thereby adds convenience to the user. As is also true in this preferred embodiment, the package preferably provides a configuration such that the composition and multiple applicators may be displayed at the same time. Such a configuration also adds convenience to the user. In one highly preferred embodiment, the package is a cosmetic compact having a mirror and a configuration such that the composition and multiple applicators are displayed at the same time. In such highly preferred embodiment, the user may use just one of the applicators and tilt the package at an angle upon application of the composition to the applicator, or upon application to the skin while looking in the mirror. During such usage, the dividing means may also prevent the remaining applicators in the package from dropping out from the package.

The personal care product of the present invention may have at least two types of compositions. The multiple applicators may be different types of applicators that are suitable for the different types of compositions. One such embodiment for a foundation and concealer is described above. Other embodiments include many other possible combinations of cosmetic products mentioned above, such as: foundation and blusher, blusher and eyeshadow, eyebrow and eyeliner, just to mention a few.

The replaceable tray and multiple applicators may be provided as a replaceable applicator kit, and provided or sold separately from the personal care product.

### Method of Making up the Skin

The present invention relates to the method of making up the skin using the present foundation and present concealer described hereinabove. Conventionally, concealers are recommended for use prior to application of the foundation. The present concealer can be used either prior to or after application of the foundation. Such “after application” is possible, because the color of the present foundation and present concealer are substantially the same. Further, the present concealer can be used as a touch up during the day.

In one highly preferred embodiment, the present foundation and present concealer are of powder form provided in the same package, such package being a compact comprising separate applicators for the foundation and concealer. Such compact may be carried and used during the day. Such delivery system provides an effective method of make up wherein the foundation and concealer are applied via different applicators, thus avoiding contamination of the compositions. Further, such delivery system is highly convenient for providing a method of make up wherein the foundation and concealer may be used independently for touch up during the day.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Powder Form Foundation Composition for Example 1

| | | |
|---|---|---|
| 1 | Talc coated with Methicone *1 | 30.36 |
| 2 | Mica coated with Methicone *2 | 35 |
| 3 | Titanium Dioxide coated with Methicone *3 | 10 |
| 4 | Methyl Methacrylate Crosspolymer *4 | 10 |
| 5 | Nylon-6 *5 | 1 |
| 6 | Yellow Iron Oxide coated with Methicone *6 | 1.81 |
| 7 | Black Iron Oxide coated with Methicone *7 | 0.2 |
| 8 | Red Iron Oxide coated with Methicone *8 | 0.43 |
| 9 | Niacinamide *9 | 0.1 |
| 10 | Panthenol *10 | 0.1 |
| 11 | Dimethicone *11 | 6.35 |
| 12 | Ethylhexyl Methoxycinnamate *12 | 4 |
| 13 | D-delta-tocopherol *13 | 0.1 |
| 14 | Perfume | 0.05 |
| 15 | Preservatives | 0.5 |
| | Total | 100 |

Definitions of Components
*1 Talc coated with Methicone: SI TALC available from Miyoshi Kasei
*2 Mica coated with Methicone: SI SERICITE available from Miyoshi Kasei
*3 Titanium Dioxide coated with Methicone: SI Titanium Dioxide IS available from Miyoshi Kasei
*4 Methyl Methacrylate Crosspolymer: GANZ PEARL GMX-0610 available from GANZ CHEMICAL CO., LTD
*5 Nylon-6: POMP610 available from Ube Kosan
*6 Yellow Iron Oxide coated with Methicone: SI MAPICO YW LIGHT LEMON XLO available from Daito Kasei
*7 Black Iron Oxide coated with Methicone: SI BLACK IRON OXIDE NO. 247 available from Daito Kasei
*8 Red Iron Oxide coated with Methicone: SI PURE RED IRON OXIDE R-3098 available from Daito Kasei
*9 Niacinamide: Niacinamide available from Reilly Industries Inc
*10 Panthenol: DL-Panthenol available from Alps Pharmaceutical Inc
*11 Dimethicone: SILICONE OIL SH200C-50CS available from Dow Corning
*12 Ethylhexyl Methoxycinnamate: PARSOL MCX available from ROCHE VITAMINS JAPAN K.K
*13 D-delta-tocopherol: D-DELTA-TOCOPHEROL available from EISAI CO., LTD.

### Method of Preparation

Component numbers 1-10 and 15 were mixed with a mixer to make a pigment component. Separately, component numbers 11-14 were mixed to make a binder component. The binder component was added into the pigment component and mixed by a mixer. The obtained composition was pressed in a pan.

### Powder Form Concealer Composition for Example 2-3

| | | Ex. 2 | Ex. 3 |
|---|---|---|---|
| 1 | Talc coated with Aluminum Dimyristate *1 | 5.74 | |
| 2 | Sericite coated with Aluminum Dimyristate *2 | 23 | |
| 3 | Mica coated with Aluminum Dimyristate *3 | 17.5 | 29.15 |
| 4 | Sericite coated with 57.3% Titanium Dioxide and | 5 | 50 |
| | Methicone *4 | | |
| 5 | Titanium Dioxide coated with Methicone *5 | 27 | |
| 6 | Methyl Methacrylate Crosspolymer *6 | 8 | |
| 7 | Yellow Iron Oxide coated with Methicone *7 | 3.84 | 5.5 |
| 8 | Black Iron Oxide coated with Methicone *8 | 0.4 | 0.48 |
| 9 | Red Iron Oxide coated with Methicone *9 | 0.82 | 1.17 |
| 10 | Niacinamide *10 | 0.1 | 0.1 |
| 11 | Panthenol *11 | 0.1 | 0.1 |
| 12 | Dimethicone *12 | 3.85 | 8.85 |
| 13 | Ethylhexyl Methoxycinnamate *13 | 4 | 4 |
| 14 | D-delta-tocopherol *14 | 0.1 | 0.1 |
| 15 | Perfume | 0.05 | 0.05 |
| 16 | Preservatives | 0.5 | 0.5 |
| | Total | 100 | 100 |

Definitions of Components
*1 Talc coated with Aluminum Dimyristate: 5MI-TALC JA-46R available from Miyoshi Kasei
*2 Sericite coated with Aluminum Dimyristate: 5MI-SERICITE available from Miyoshi Kasei
*3 Mica coated with Aluminum Dimyristate: 5MI-MICA M-102 available from Miyoshi Kasei
*4 Sericite coated with 57.3% Titanium Dioxide and Methicone: Fancyveil S-3060SW available from Shokubai Kasei
*5 Titanium Dioxide coated with Methicone: SI Titanium Dioxide IS available from Miyoshi Kasei
*6 Methyl Methacrylate Crosspolymer: GANZ PEARL GMX-0610 available from GANZ CHEMICAL CO.
*7 Yellow Iron Oxide coated with Methicone: SI MAPICO YW LIGHT LEMON XLO available from Daito Kasei
*8 Black Iron Oxide coated with Methicone: SI BLACK IRON OXIDE NO. 247 available from Daito Kasei
*9 Red Iron Oxide coated with Methicone: SI PURE RED IRON OXIDE R-3098 available from Daito Kasei
*10 Niacinamide: Niacinamide available from Reilly Industries Inc
*11 Panthenol: DL-Panthenol available from Alps Pharmaceutical Inc
*12 Dimethicone: SILICONE OIL SH200C-50CS available from Dow Corning
*13 Ethylhexyl Methoxycinnamate: PARSOL MCX available from ROCHE VITAMINS JAPAN K.K
*14 D-delta-tocopherol: D-DELTA-TOCOPHEROL available from EISAI CO., LTD.

### Method of Preparation

Component numbers 1-11 and 16 were mixed with a mixer to make a pigment component. Separately, component numbers 12-15 were mixed to make a binder component. The binder component was added into the pigment component and mixed by a mixer. The obtained composition was pressed in a pan.

### Liquid Form Foundation Composition for Example 4

| | | |
|---|---|---|
| 1 | Titanium Dioxide coated with Dimethicone *1 | 10 |
| 2 | Vinyl Dimethicone/Methicone Silesquioxane | 3.45 |
| | Crosspolymer *2 | |
| 3 | Yellow Iron Oxide coated with Methicone *3 | 1.3 |
| 4 | Black Iron Oxide coated with Methicone *4 | 0.1 |
| 5 | Red Iron Oxide coated with Methicone *5 | 0.15 |
| 6 | Sorbitan Monoisostearate *6 | 5 |
| 7 | Dimethicone Copolyol *7 | 20 |
| 8 | Cyclopentasiloxane *8 | 29.85 |
| 9 | D-delta-tocopherol *9 | 0.1 |
| 10 | Water | 16.3 |
| 11 | Mulberry Root Extract *10 | 3 |
| 12 | Saccharomycopsis Ferment Filtrate *11 | 5 |
| 13 | Niacinamide *12 | 0.1 |
| 14 | Panthenol *13 | 0.1 |
| 15 | Butylene Glycol *14 | 5 |
| 16 | Perfume | 0.05 |
| 17 | Preservatives | 0.5 |
| | Total | 100 |

Definitions of Components
*1 Titanium Dioxide coated with Dimethicone: SA Titanium Dioxide IS available from Miyoshi Kasei
*2 Vinyl Dimethicone/Methicone Silesquioxane Crosspolymer: KSP-100 available from Shinetsu Silicone
*3 Yellow Iron Oxide coated with Methicone: SI MAPICO YW LIGHT LEMON XLO available from Daito Kasei
*4 Black Iron Oxide coated with Methicone: SI BLACK IRON OXIDE NO. 247 available from Daito Kasei
*5 Red Iron Oxide coated with Methicone: SI PURE RED IRON OXIDE R-3098 available from Daito Kasei
*6 Sorbitan Monoisostearate: Crill 6 available from Croda
*7 Dimethicone Copolyol: DC5225C available from Dow Corning
*8 Cyclopentasiloxane: SH245 available from Dow Corning
*9 D-delta-tocopherol: D-DELTA-TOCOPHEROL available from EISAI CO., LTD.
*10 Mulberry Root Extract: Mulberry BG, available from Maruzen Pharmaceuticals
*11 Saccharomycopsis Ferment Filtrate: SK-II Pitera available from Kashiwayama
*12 Niacinamide: Niacinamide available from Reilly Industries Inc
*13 Panthenol: DL-Panthenol available from Alps Pharmaceutical Ind
*14 Butylene Glycol: 1,3 Butylene Glycol available from Kyowa Hakko Kogyo

### Method of Preparation

Component numbers 6-9 and 16 were mixed with suitable mixer until homogeneous to make a lipophilic mixture. Component numbers 1-5 were mixed with suitable mixer until homogeneous to make a powder mixture. The powder mixture was pulverized using pulverizer. The powder mixture was added into the lipophilic mixture with suitable mixer until homogeneous. Component numbers 10-15 and 17 were dissolved with suitable mixer until all components were completely dissolved to make a water phase. The water phase was added into the product of powder added lipophilic mixture to effect emulsion at room temperature using homogenizer.

### Liquid Form Concealer Composition for Example 5

| | | |
|---|---|---|
| 1 | Titanium Dioxide coated with Dimethicone *1 | 25 |
| 2 | Sericite coated with 57.3% Titanium Dioxide and | 10 |
| | Methicone *2 | |
| 3 | Yellow Iron Oxide coated with Methicone *3 | 6.08 |
| 4 | Black Iron Oxide coated with Methicone *4 | 0.28 |
| 5 | Red Iron Oxide coated with Methicone *5 | 0.39 |
| 6 | Sorbitan Monoisostearate *6 | 3 |
| 7 | Dimethicone Copolyol *7 | 15 |
| 8 | Isotridecyl Isononanoate *8 | 11.85 |
| 9 | D-delta-tocopherol *9 | 0.1 |
| 10 | Water | 21.05 |
| 11 | Mulberry Root Extract *10 | 1.5 |
| 12 | Saccharomycopsis Ferment Filtrate *11 | 2.5 |
| 13 | Niacinamide *12 | 0.1 |
| 14 | Panthenol *13 | 0.1 |
| 15 | Butylene Glycol *14 | 2.5 |
| 16 | Perfume | 0.05 |
| 17 | Preservatives | 0.5 |
| | Total | 100 |

Definitions of Components
*1 Titanium Dioxide coated with Dimethicone: SA Titanium Dioxide IS available from Miyoshi Kasei
*2 Sericite coated with 57.3% Titanium Dioxide and Methicone: Fancyveil S-3060SW available from Shokubai Kasei
*3 Yellow Iron Oxide coated with Methicone: SI MAPICO YW LIGHT LEMON XLO available from Daito Kasei
*4 Black Iron Oxide coated with Methicone: SI BLACK IRON OXIDE NO. 247 available from Daito Kasei
*5 Red Iron Oxide coated with Methicone: SI PURE RED IRON OXIDE R-3098 available from Daito Kasei
*6 Sorbitan Monoisostearate: Crill 6 available from Croda
*7 Dimethicone Copolyol: DC5225C available from Dow Corning
*8 Isotridecyl Isononanoate: Crodamol TN available from Croda
*9 D-delta-tocopherol: D-DELTA-TOCOPHEROL available from EISAI CO., LTD.
*10 Mulberry Root Extract: Mulberry BG, available from Maruzen Pharmaceuticals
*11 Saccharomycopsis Ferment Filtrate: SK-II Pitera available from Kashiwayama
*12 Niacinamide: Niacinamide available from Reilly Industries Inc
*13 Panthenol: DL-Panthenol available from Alps Pharmaceutical Ind
*14 Butylene Glycol: 1,3 Butylene Glycol available from Kyowa Hakko Kogyo

### Method of Preparation

Component numbers 6-9 and 16 were mixed with suitable mixer until homogeneous to make a lipophilic mixture. Component numbers 1-5 were mixed with suitable mixer until homogeneous to make a powder mixture. The powder mixture was pulverized using pulverizer. The powder mixture was added into the lipophilic mixture with suitable mixer until homogeneous. Component numbers 10-15 and 17 were dissolved with suitable mixer until all components were completely dissolved to make a water phase. The water phase was added into the product of powder added lipophilic mixture to effect emulsion at room temperature using homogenizer.

### Usage

Product 1 comprises the powder form foundation of Example 1 and powder form concealer of Example 2. Product 2 comprises the powder form foundation of Example 1 and powder form concealer of Example 3. Example 1 is useful as a powder form foundation having appropriate sheer coverage. By applying to the whole face, the foundation provides uniform skin tone and texture. Examples 2 and 3 are useful as powder form concealers having high coverage and are partially applied on skin defects such as spots.

Product 3 comprises the liquid form foundation of Example 4 and liquid form concealer of Example 5. Example 4 is a liquid form foundation having appropriate sheer coverage. By applying to the whole face, the foundation provides uniform skin tone and texture. Example 5 is a liquid form concealer having high coverage and is partially applied on skin defects such as spots.

The color difference (CMC DE) between the foundation and concealer of Products 1 through 3, respectively, were measured by colorimeter as provided below. For Examples 1 through 3, the cake surface was measured. For Examples 4 and 5, bulk surface wrapped by thin transparent film was measured.

| | | |
|---|---|---|
| Product 1 | Product 2 | Product 3 |

| | | | | | | |
|---|---|---|---|---|---|---|
| No. of Example | Ex. 1 | Ex. 2 | Ex. 1 | Ex. 3 | Ex. 4 | Ex. 5 |

| | | | |
|---|---|---|---|
| CMC DE | 0.78 | 0.17 | 0.11 |

The color difference (CMC DE) between the foundation and concealer of Products 1 through 3 were within 1.5. Products 1 through 3, regardless of whether the concealer is applied before or after foundation, provides seamless appearance by having similar color and texture, and without giving unnaturalness between foundation and concealer.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic product comprising:
(a) a foundation comprising less than about 15% by weight of the foundation of a high coverage pigment selected from the group consisting of titanium dioxide, zinc oxide, and mixtures thereof; and
(b) a concealer comprising at least about 25% by weight of the concealer of a high coverage pigment selected from the group consisting of titanium dioxide, zinc oxide, and mixtures thereof; wherein;
(c) the CMC DE of the foundation color and the concealer color are less than 1.5; and
(d) the foundation and concealer are of the same product form.

2. The cosmetic product of claim 1 wherein the foundation and concealer are of a powder form.

3. The cosmetic product of claim 2 wherein the foundation and concealer are provided in the same package.

4. The cosmetic product of claim 3 wherein multiple applicators are provided in the same package.

5. The cosmetic product of claim 4 wherein at least two types of applicators are provided in the same package.

6. The cosmetic product of claim 4 wherein the applicators are housed in a replaceable tray which engages with the package.

7. A replaceable applicator kit for the cosmetic product of claim 6 wherein the replaceable applicator kit comprises the replaceable tray and multiple applicators.

8. The cosmetic product of claim 1 wherein the foundation and concealer are of a liquid form.

9. A method of making up the skin comprising the steps of:
(1) applying to the skin the foundation of claim 1; and
(2) applying to the skin the concealer of claim 1.

10. A personal care product comprising:
(a) a composition for care of a personal surface;
(b) multiple applicators for applying the composition to a personal surface; and
(c) a replaceable tray for housing the multiple applicators;
wherein the above mentioned elements are provided in the same package.

11. A personal care product comprising:
(a) a composition for care of a personal surface;
(b) multiple applicators for applying the composition to a personal surface; and
(c) a dividing means for keeping each of the multiple applicators in a designated position;
wherein the above mentioned elements are provided in the same package.

12. The personal care product of claim 10 wherein the package provides a configuration such that the composition and multiple applicators are displayed at the same time.

13. The personal care product of claim 10 wherein at least two types of applicators are provided.

14. The personal care product of claim 10 wherein at least two types of compositions are provided.

15. A replaceable applicator kit for the personal care product of claim 10 wherein the replaceable applicator kit comprises the replaceable tray and multiple applicators.
